(19) United States
(12) Patent Application Publication
(10) Pub. No.: US 2014/0221913 A1
(43) Pub. Date: Aug. 7, 2014

(54) LOW PROFILE INFUSION PUMP WITH ANTI DRUG DIVERSION AND ACTIVE FEEDBACK MECHANISMS

(71) Applicant: MEDIPACS, INC., Tucson, AZ (US)

(72) Inventors: Mark Banister, Tucson, AZ (US); Raymond Clark, Tucson, AZ (US); Erich Coiner, Tucson, AZ (US); Yordan M. Geronov, Tucson, AZ (US); Mark D. McWilliams, San Diego, CA (US); Ralph Sias, Tucson, AZ (US); Mark Van Veen, Tucson, AZ (US); Gary Walters, Tucson, AZ (US)

(73) Assignee: Medipacs, Inc., Tucson, AZ (US)

(21) Appl. No.: 14/347,597

(22) PCT Filed: Sep. 25, 2012
(86) PCT No.: PCT/US2012/057129

§ 371 Date: Mar. 26, 2014

Related U.S. Application Data
(60) Provisional application No. 61/539,339, filed on Sep. 26, 2011.

(51) Int. Cl. A61M 5/142; A61M 5/168; A61M 5/172

(52) U.S. Cl. CPC A61M 5/14248; A61M 5/1723; A61M 5/16881; A61M 2005/1726; A61M 2205/0266; A61M 2205/0283; A61M 2205/0294; A61M 2205/3317; A61M 2205/3331; A61M 2205/3368; A61M 2205/502; A61M 2209/045; A61M 2230/201; A61M 2230/205
USPC 604/66; 604/151; 604/134; 604/150; 604/140; 604/67

(57) ABSTRACT

A low profile and volumetrically efficient medication delivery device configured to be placed on the body of a patient during fluid delivery to the patient is provided. The device incorporates a low profile actuator assembly that causes fluid delivery by displacing a collapsible reservoir in response to receiving an electrical current or charge input from a programmable controller and a power supply. Additional components help prevent drug theft or abuse, measure actuator pressure, displacement and temperature in real time providing active feedback to the controller that is controlling the actuator. The device also has wireless communication capabilities.

The present invention concerns a low profile and volumetrically efficient medication delivery device configured to be placed on the body of a patient during fluid delivery to the patient. More particularly the present invention incorporates a low profile actuator assembly that causes fluid delivery by displacing a collapsible reservoir in response to receiving an electrical current or charge input from a programmable controller and a power supply. Additional components in the present invention help prevent drug theft or abuse, measure actuator pressure, displacement and temperature in real time providing active feedback to the controller that is controlling the actuator. The device also has wireless communication capabilities.

Patients have need of pumped medication in a variety of settings. Most pumps such as IV pumps are designed to deliver large volumes of fluids making them relatively bulky and usually only suitable at a bedside in a managed care environment such as a hospital. More recently medications such as insulin have been delivered by more portable pumps that may be fastened to the body. Such pumps are more convenient than the larger IV pumps but still suffer from tradeoffs such as complexity, high cost and volume limitations on the amount of medication that may be carried and delivered. Moreover, wearing a pump on the body can be quite uncomfortable. There is a need to improve upon these pumps in terms of their complexity, cost and efficiency.

Drug diversion or theft is a major problem in the healthcare industry today. Drug pump manufacturers today rely on the facilities using the pumps to maintain drug controls. The present inventions addresses this problem by making it difficult to access the drug once it is loaded into and stored inside of the device. The exterior chassis provides a secure shell around the drug once the device is filled, then utilizing bends in fluid channels, anti siphon valves, one way valves and collapsible tubes, as well as wireless technologies such as RFID, IR, Bluetooth and Zigby to provide location information and alarms if device is tampered with or removed from area. Furthermore one or more real time sensor feedback loops allows the device to accurately control the delivery rates produced from the positive displacement actuator that meters or delivers the drug from the drug container to the patient.

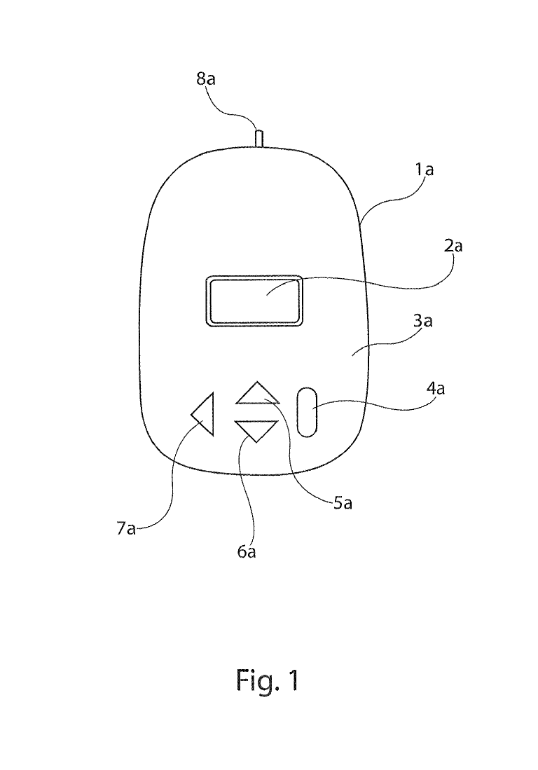

FIG. 1 depicts an exemplary frontal view of the medication delivery device of the present invention.

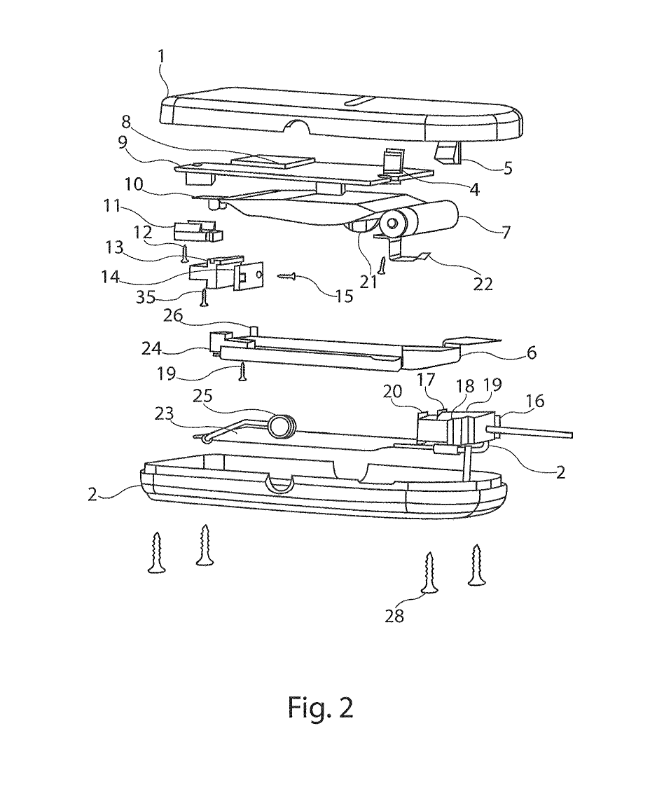

FIG. 2 depicts a medication delivery device assembly utilized in the medication delivery device of the present invention.

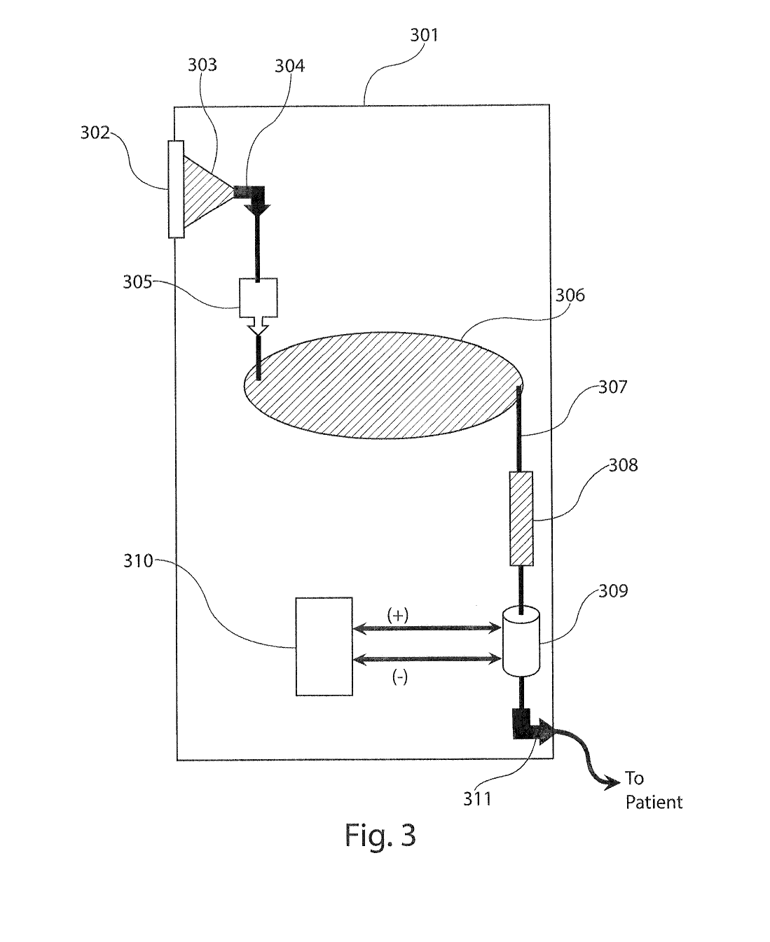

FIG. 3 depicts one example of the fluidic circuit, anti-diversion valves and other fluidic components within the rigid exterior case.

The foregoing refers to geometric terms such as “upper”, “lower”, and “side.” These are not to imply or be limited to a direction with respect to a gravitational reference frame but are utilized to distinguish directions relative to each other. Some details in the various embodiments such as how current or wiring is routed to electrodes from power supplies are left out for illustrative simplicity since some of these methods of such routing are known in the art. The term Electrolyte refers to and includes all aqueous, non aqueous, polymer and solid electrolytes, including those that are generally well known in the art. The term electrodes refer to anodes and cathodes commonly used in electrochemical systems that are made of materials well known in the art such as metals, carbons, graphenes, oxides or conducting polymers. The term separator refers to any nano, micro or macro porous material that allows targeted ions to move through or across it faster than surrounding ion containing media. The term ion refers to ions and ion species as well as anion, cation, electrons & protons. The term housing refers to the exterior portion of the device which may be fabricated from flexible material, rigid material, elastic materials, non elastic materials or a combination of these such as rubbers, silicone, polyurethane, metalized polymer films and other plastics or polymers known in the art. The housing is configured to allow movement and expansion of the internal parts as well as allowing for filling device with electrolyte, acting as a container and barrier to stop any electrolyte leakage or evaporation. The housing may also the ability to vent any unwanted gas generation if needed.

The foregoing refers to polymeric actuators. In an exemplary embodiment these polymer actuators are formed from pH responsive epoxy polymer Hydrogel based polymers. Examples of such polymers are described in commonly owned WIPO patent application WO2008079440A2, Entitled “SUPER ELASTIC EPOXY HYDROGEL”, filed on Jul. 10, 2007 and published on Jul. 3, 2008. other polymer actuator examples may contain polymers which have ionic functional groups, such as carboxylic acid, phosphoric acid, sulfonic acid, primary amine, secondary amine, tertiary amine, and ammonium, Acrylic acid, methacrylic acid, vinylacetic acid, maleic acid, metakuriro yloxy ethylphosphoric acid, vinylsulfonic acid, styrene sulfonic acid, Vinylpyridine, vinylaniline, vinylimidazole, aminoethyl acrylate, Methylamino ethyl acrylate, dimethylamino ethyl acrylate, Ethylamino ethyl acrylate, ethyl methylamino ethyl acrylate, Diethylamino ethyl acrylate, aminoethyl methacrylate, Methylamino ethyl methacrylate, dimethylaminoethyl methacrylate, Ethylamino ethyl methacrylate, ethyl methylamino ethyl methacrylate, Diethylamino ethyl methacrylate, aminopropyl acrylate, Methylaminopropyl acrylate, dimethylaminopropylacrylate, Ethylaminopropyl acrylate, ethyl methylaminopropyl acrylate, Diethylamino propylacrylate, aminopropyl methacrylate, methylaminopropyl methacrylate, dimethylaminopropyl methacrylate, ethylaminopropyl methacrylate, ethyl methylaminopropyl methacrylate, Polymers, such as diethylamino propyl methacrylate, dimethylaminoethyl acrylamide, dimethylaminopropylacrylamide, and akuriroyloxy ethyl trimethylammonium salts, are reported to be of use but these examples are for reference and not intended to limit the scope or use of the invention.

The actuator mechanism used to dispense the contents of the drug reservoir can be as simple as a spring that applies pressure to a pivot plate pressing on the collapsible drug reservoir, or as complex as a stepper motor or actuator turning a lead screw mechanism that in turn pushes a plunger in a syringe or cylindrical type of reservoir to dispense the reservoir contents. In the case of a non electronically controlled positive pressure mechanism such as a spring an electronically controlled valve is needed. In the case of an electronically controlled actuator, non electronic pressure valves can suffice for the system to work properly. A combination of both types of valves for either types of actuation would be the preferred embodiment to present the best protection from drug diversion or theft.

The anti diversion valves work in conjunction with any mechanical or non mechanical method used to displace and dispense the contents of the drug chamber or reservoir of the pump as long as they are in fluidic connectivity and in line between the fill point and the drug reservoir, and again in fluidic connectivity and in line between the exit point and the fluid reservoir. When housed or protected within an unbreakable or difficult to break exterior case, the combination of the case, fluidic circuit and the anti diversion valves make it very difficult for an un authorized person to remove the drug contents from the fill or inlet opening and fluid path as well as the outlet opening and exit fluid path without destroying the device and setting off alarms. Materials used in the septum fill port and valve and the exit point and valves are made of plastics, metals or other materials that are impervious to penetration with sharp needles or other implement that would allow free flow access to the contents of the device. These examples are for reference and not intended to limit the scope or use of the invention.

The sensors incorporated into the device and controller loop may be internal or external sensors as well as a combination of the two. The sensors may determine patient physiology needs or conditions an example would be a glucose sensor to help regulate the delivery of insulin to a patient. Another example would be a proximity sensor used to transmit location of the device for safety of the patient and theft deterrent. Other sensor uses include measuring the volume displacement of the drug reservoir or the actuator displacement. There are many sensors that can accomplish this such as Hall Effect sensors, another example would be electrical resistance across the interior or exterior of the drug container, actuator or both. Flow rate and pressure sensors are particularly useful as feedback to the pump controller to confirm performance and accuracy of the reservoir displacement by the actuating mechanism as well as determining valve control or timing for accurate delivery of the medication.

In one aspect of the invention there is provided: a low profile medication delivery device comprising a chassis having an internal fluid reservoir and internal pump mechanism for pumping fluid from an internal fluid reservoir, wherein the internal reservoir has a fluid inlet accessible from an exterior of the chassis for filling the internal fluid reservoir, and an inlet valve in fluid connectivity between the inlet and the reservoir; and a fluid outlet exiting the chassis, and one or more exit valves in fluid connectivity between the reservoir and the fluid outlet.

In one embodiment the inlet comprises a rigid inlet fluid channel with one or more bends totaling at least 90°, or bends sufficient to impede access by a needle or other object,

In another embodiment the inlet valve comprises an one way anti siphon valve.

In yet another embodiment the inlet is covered by a septum that can be pierced, and the septum is held in place by a rigid housing with an internal fluid channel in fluid connectivity with the inlet valve.

In one embodiment the fluid outlet comprises a rigid fluid channel with one or more bends totaling at least 90°, or bends sufficient to impede access by a needle or other object, in fluid connectivity between the reservoir and the outlet valve.

In another embodiment, the device further comprises a second collapsible tube valve between the reservoir and the outlet valve.

In one embodiment, the pump mechanism is electrically powered and has a power source and controller.

If desired, the outlet valve may be electrically controlled via the controller.

In another embodiment the pump mechanism is selected from a spring, a piezo actuator, a nitenol or shape memory material, a hydraulic actuator, and a pneumatic actuator.

In one embodiment the outlet valve is adapted to open and close from changes in pressure.

In yet another embodiment the device further comprises one or more actuators and one or more reservoirs to hold and dispense one or more medications.

In another aspect of the invention there is provided a low profile medication delivery device comprising: a chassis; and an actuator overlaying a collapsible reservoir disposed within the chassis. The collapsible reservoir has an inlet and outlet accessible from an exterior of the chassis, and electronics disposed within the chassis in electrical connection with the actuator, the actuator configured to expand and compress the collapsible reservoir in response to electrical current being passed through the actuator. One or more sensors are electrically connected to the electronics; a power supply is provided in electrical connection with the electronics, and a controller to control the pump, sensors and electronics; and a septum and valve are provided in fluid connection with the reservoir inlet and one or more outlet valves in fluid connection with the reservoir outlet to control fluid being pumped as the collapsible reservoir is compressed.

In one embodiment the actuator includes an anode and a cathode, a polymeric actuator material between the anode and cathode, an ionic separator membrane separating the anode or cathode from the polymer actuator material, an electrolyte and a flexible exterior housing.

In another embodiment the actuator includes an activation mechanism selected from the group consisting of a spring, a piezo actuator, a nitenol or shape memory material, a hydraulic actuator, and an pneumatic actuator.

In yet another embodiment the one or more sensors are selected from the group consisting of a motion sensor, a temperature sensor and a pressure sensor for sensing movement, temperature or pressure generated by the actuator.

In still yet another embodiment one or more of the sensors provide external feedback from the patient to the controller.

In one embodiment the device further comprises a rigid plate located between the actuator and the fluid reservoir. If desire, the rigid plate may have one or more hinges or flexure arms.

In yet another embodiment the device further includes a magnet located on the plate in close proximity to a magnorestrictive sensor to measure movement of the plate.

In still yet another embodiment the outlet valve is controlled to open and close from changes in pressure.

In one embodiment the outlet valve is electrically controlled via the controller

In yet another embodiment the device comprises one or more actuators and one or more reservoirs to hold and dispense one or more medications.

In still yet another embodiment the inlet comprises a rigid inlet fluid channel with one or more bends of at least 90° in fluid connectivity between the inlet and the inlet valve.

If desired in yet another embodiment the inlet valve is a one way anti siphon valve.

In one embodiment the outlet comprises a rigid fluid channel with one or more bends of at least 90° in fluid connectivity between the reservoir and the outlet valve.

If desired, the device may further comprise a second collapsible tube valve between the reservoir and the outlet valve.

In still yet another embodiment of the invention there is provided a low profile medication delivery device comprising: a chassis and an actuator overlaying a collapsible reservoir disposed within the chassis, the collapsible reservoir having an inlet and outlet that exit the chassis. The actuator is configured to expand and compress the collapsible reservoir in response to electrical current being passed through the actuator. A user interface is provided for providing input to the electronics disposed within the chassis in electrical connection with the actuator, and one or more sensors electrically connected to the electronics. A power supply is in electrical connection with the electronics, and a controller to control the pump, sensors and electronics. Finally, a septum and valve are in fluid connection with the reservoir inlet and one or more outlet valves in fluid connection with the reservoir outlet to control the fluid being pumped as the collapsible reservoir is compressed.

In one embodiment the user interface is adapted to provide input information to the controller and visual information to the user.

In another embodiment the controller is programmable and the program includes software hardrails that prevent the device pumping or dispensing a lethal dose of medication to the patient, whether the lethal dose is accumulated over time or all at once.

In yet another embodiment the actuator includes an anode and a cathode, and has a polymeric actuator material between the anode and cathode, an ionic separator membrane separating the anode or cathode from the polymer actuator material, an electrolyte and a flexible exterior housing.

In still yet another embodiment the actuator includes an actuation mechanism selected from the group consisting of a spring, a pies actuator, a nitenol or shape memory material, a hydraulic actuator, and a pneumatic actuator.

In one embodiment the one or more sensors are selected from the group consisting of a motion sensor, a temperature sensor and a pressure sensor.

In another embodiment one or more of the sensors provide external feedback from the patient to the controller.

In still yet another embodiment the device includes a rigid plate located between the actuator and the fluid reservoir. If desired, the rigid plate may have one or more hinges or flexure arms, and optionally may further include a magnet located on the plate in close proximity to a magnorestrictive sensor to measure movement of the plate.

In one embodiment the outlet valve is controlled to open and close from changes in pressure.

In another embodiment the outlet valve is electrically controlled via the controller.

In still yet another embodiment the device comprises one or more actuators and one or more reservoirs to hold and dispense one or more medications.

In one embodiment the inlet comprises a rigid inlet fluid channel with one or more bends of at least 90° in fluid connectivity between the inlet and the inlet valve.

In another embodiment the inlet valve is a one way anti siphon valve.

In yet another embodiment the outlet comprises a rigid fluid channel with one or more bends of at least 90° in fluid connectivity between the reservoir and the outlet valve. If desired, the device further comprises a second collapsible tube valve between the reservoir and the outlet valve.

Electrically controlled valves add an even greater level of security as the pump controller can open and close them according to controller software algorithms for the filling of the device and subsequent delivery of the medication to the patient. with the valves staying closed or locked anytime other than when the device is in use and shut them if there is any tampering with the device.

An exemplary embodiment of a medication delivery apparatus according to the present invention is depicted in diagram form with respect to FIG. 1. Shown are the exterior chassis (1a), disposed upon exterior chassis is a user interface. User interface may include any or all of input keys or buttons, display and LED indicator lights. The user interface may also include a wireless device (not shown) utilized to control medication delivery device, and facilitate access to electronic medical records stored in the device memory. The user interface is also configured to facilitate secure access to the device when necessary. Electronic display screen (2a), LED light port (3a) that indicates a visual state of device via color and on time as controlled by the controller and software, on/off button (4a), menu up button (5a), menu down button (6a), menu select button (7a) the functionality and number of the buttons can be changed via programming and their number and description is not meant to limit the applications and functionalities of the user interface components. FIG. 1 also shows tubing exiting the device (8a) to deliver fluid to the patient via subcutaneous, subdermmal, IV route or other method to cross the skin barrier and allow fluid connectivity of the fluid reservoir, containing medication or other liquids, to the patient.

Another preferred embodiment of the pump apparatus is shown in FIG. 2 and includes an exterior chassis top half (1), exterior chassis bottom half (2), when assembled they are configured to be attached to, mounted to a patients body and can even be held in a strap like holster that holds the device comfortably on a patients body. These components are typically plastic injection molded parts and this process is very well known in the art. Within the exterior chassis is a collapsible reservoir (23) adjacent to a pivot plate (6) adjacent to an actuator assembly (10) that is electrically coupled to controller, memory electronics, wireless, (9) and power source (7). All assembled components are held together by screws such as (12, 15, 19, 28, and 35) where needed or plastic welding such as but not limited to RF or ultrasonic welding, glues and epoxies may also be used where needed. Again all these materials and components and how to manufacture them, with the exception of the actuator polymer material, are well known in the art and could easily be manufactured by anyone experienced in the art.

In a preferred embodiment the actuator assembly (10) is responsive to electrical current received from control electronics (9). The actuator assembly (10) is configured to expand and press upon pivot plate (6) the pivot plate in turn presses against the collapsible reservoir (23). The pivot plate is hinged and the hinges attach the exterior chassis in order to restrict movement to one plane of direction. To determine the amount of movement or position of the pivot plate, a magneto restrictive or other type sensor with electronics (14) is mounted on the circuit board and another sensor, magnet (26), mounting plate (13) or other sensor readable surface is attached to the pivot plate in such a way that when the pivot plate moves the sensor can read the movement as the sensor, magnet or sensor reading surface passes the sensor. The sensor in turn sends a signal, of the movement, back to the controller so the controller can increase, decrease, extend or turn off the electrical current it is sending to the actuator. The fill port or septum is shown (25) and exit solenoid valve (17), manifold (18) and check valve (19) with mounting bracket assembly (20) is also shown.

A pressure or force sensor (16) is used to track the operating pressure and provide feedback to the controller and an audible alarm (21) is also connected to the electronics that will provide audible alert if there is a problem with the pump or if it has finished delivering all of the fluids. The electronics also include a wireless chip set in connectivity so that programming and record information as well as location and can be sent to and from the device wirelessly. The electronics board also include battery clips (4, 5) and sensor or other mounting brackets (11, 14, 22) the circuitry, chips, sensors LEDs and related electronics components, board and power source.

For the filling of the reservoir and to deter drug diversion one example of the fluid circuit is shown in FIG. 3 and encased in a hard rigid exterior case (301), a closed pierce able septum valve (302) utilizing a hard or rigid holder or encasement with a reducing shaped internal opening behind the septum (303) and a degree of bending needed to stop a needle or tube from being inserted down the tubing elbow (304), or “S” bend in the fluid channel. These are included to stop any needle or other device from traveling past or piercing the tubing beyond the septum holder in order to access the inlet tubing, one way valve or collapsible fluid reservoir.

The inlet fluid channel tubing leading to the collapsible reservoir (306) incorporates a one way valve (305) that allows fluid to enter from the septum valve but will not allow fluid to be with drawn back through the septum valve. One way valves known to industry are many and well known in the art. Examples include reed valves, ball valve, piston valves, check valves, electric solenoid valves, etc. and are included for descriptive purposes of the many types that may be used alone or in conjunction with other valves and components in the fluid circuit to make drug diversion or theft from the invention very difficult.

To stop drug diversion at the fluid exit point of the device, while the device is in operation mode delivering the drug to the patient, while in operation the controller (310) controls the open and close timing of the shutoff valve via electrical current. In other examples the shutoff valve may be non electrical and only pressure driven, these are only a couple of examples of the many types of shutoff valves that could be used and are well known in the industry. There is a secondary system, between the fluid reservoir (306) and the shutoff valve (309), a collapsible fluid channel, tube or anti siphon check valve (308) is located in fluid connection to the exit tubing (307), this valve will close if fluid is drawn faster than the flow rate of the fluid exiting the open shut off valve (309) and interrupt the fluid flow out of the fluid reservoir. This stops drawing of fluid from the device at a faster rate than the device is actually delivering to the patient while the exit valve is open for the purpose of treating the patient. Between the collapsible channel and the shutoff valve, or after the shutoff valve there is a degree of single or multiple bends such as an elbow (311) or “S” bend in the fluid channel made of rigid material to stop a needle or other thin object from being inserted into the fluid channel in order to access the collapsible reservoir while the shutoff valve is open thereby denying direct access to the fluid reservoir during pump operation.

When the pump is not actively delivering the drug to the patient the shutoff valve is closed and denies access to the contents of the reservoir in the device. When the shutoff valve is open fluid is delivered to the patient via the exit tubing. The shutoff valve can be one of many valves known to industry are many and well known in the art. Examples include reed valves, ball valve, piston valves, check valves, electric solenoid valves, pressure driven valves, only opening once the internal pressure of the reservoir hits a certain pressure or force etc. and are included for descriptive purposes of the many types that may be used in alone or in conjunction with other valves and components to make drug diversion or theft from the invention very difficult.

In the preferred embodiment the fluid shutoff valve is controlled by the electronic controller, related circuitry and input from sensors, the sensors may be located either in the device or on the patient or a combination of both, an example of this would be a pulse oximetry sensor on the patient measuring oxygen levels in a patients blood if the oxygen level falls to low, the pump can adjust to the patients needs by reducing the drug amount or increasing the drug amount dependent on the therapy and drug in use. Another example would be use of a glucose sensor to provide information to the pumps controller so it can adjust the dosage of insulin it is delivering.

In another preferred embodiment the device FIG. 2 has its longest dimension along a primary lateral axis X, a second longest dimension along secondary lateral axis Y, and the minimum dimension along depth axis Z (FIG. 3C). Axes X, Y, and Z are mutually orthogonal. This arrangement minimizes a depth dimension of device FIG. 2. This is important for patient comfort, since patients are more sensitive to the thickness of a body carried device than to the lateral dimensions.

An alternative embodiment of device FIG. 2. is a device deploying two or more reservoir/actuator assemblies for dispensing two or more different fluids to a patient. An actuator overlaying a collapsible reservoir containing a first fluid can be positioned next to or on top of another reservoir/actuator assembly containing a second fluid that is different than the first fluid. Fluids from the reservoirs exit to the patient through fluid conduits either separately or mixed together. Fluid conduits may combine to mix fluids prior to dispensing to the patient, or they may remain unmixed in individual fluid conduits.

An exemplary example of this is a device that delivers morphine to a patient, if the patient went into respiratory distress, one or mores sensors such as respiratory sensor, oximetry sensor or combination on the patient and in connectivity with the pump controller would detect that condition via low blood oxygen content and breathing. The pump would then deliver an antidote such as Naloxone in the case of morphine. The Naloxone held in a second reservoir/actuator assembly in the same device or additional device and delivers the Naloxone to the patient according to the patients sensor monitored condition. The collapsible reservoir or syringe, user interface, sensors and other features therein may be similar to those discussed with respect to FIGS. 1 and 2, but with the addition of algorithms to determine the internal or external sensor input, sensor connection and communication this can be wireless or hard wired to the device (not shown) depending where the external sensor is located on or in a patient and therapeutic conditions that meet the need for an antidote or other drug to be delivered, or therapeutic drug stopped.

Other actuator assemblies can be envisioned using multiple actuators with other designs of pivot plates, such as flexure plates, floating plates or other types and combinations of polymeric actuators. These alternative designs may be of various geometric shapes and sizes and be substituted into the medication delivery device of the present invention or used singularly, in tandem or in parallel with multiple devices and therapies wherein the devices a capable of communicating with each other and adjusting to the patients conditions automatically without departing from the broad scope of the invention.

Other embodiments can be envisioned with other arrangements and configurations of fluid bags, fluid syringes, sensors, and actuators. The scope of the present invention is not intended to be limiting except as recited in the claims.

1-43. (canceled)

44. A low profile medication delivery device comprising:
a chassis having an internal fluid reservoir and internal pump mechanism for pumping fluid from an internal fluid reservoir,
wherein the internal reservoir has a fluid inlet accessible from an exterior of the chassis for filling the internal fluid reservoir, and an inlet valve in fluid connectivity between the inlet and the reservoir; and
a fluid outlet exiting the chassis, and one or more exit valves in fluid connectivity between the reservoir and the fluid outlet.

45. The device of claim 44, wherein the inlet comprises a rigid inlet fluid channel with one or more bends of at least 90°, and/or wherein the inlet valve comprises an one way anti siphon valve.

46. The device of claim 44, wherein the inlet is covered by a septum that can be pierced,
and the septum is held in place by a rigid housing with an internal fluid channel in fluid connectivity with the inlet valve.

47. The device of claim 44, wherein the fluid outlet comprises a rigid fluid channel with one or more bends sufficient to impede access by a needle, preferably totaling at least 90° in fluid connectivity between the reservoir and the outlet valve, and optionally further comprising a second collapsible tube valve between the reservoir and the outlet valve.

48. The device of claim 44, characterized by one or more of the following features:
(a) wherein the pump mechanism is electrically powered and has a power source and controller, and wherein the outlet valve preferably is electrically controlled via the controller;
(b) wherein the pump mechanism is selected from a spring, a piezo actuator, a nitenol or shape memory material, a hydraulic actuator, and a pneumatic actuator;
(c) wherein the outlet valve is adapted to open and close from changes in pressure; and
(d) further optionally comprising one or more actuators and one or more reservoirs to hold and dispense one or more medications.

49. A low profile medication delivery device comprising:
a chassis;
an actuator overlaying a collapsible reservoir disposed within the chassis;
wherein the collapsible reservoir has an inlet and outlet accessible from an exterior of the chassis,
and electronics disposed within the chassis in electrical connection with the actuator, the actuator configured to expand and compress the collapsible reservoir in response to electrical current being passed through the actuator;
one or more sensors electrically connected to the electronics;
a power supply in electrical connection with the electronics, and a controller to control the pump, sensors and electronics; and
a septum and valve in fluid connection with the reservoir inlet and one or more outlet valves in fluid connection with the reservoir outlet to control fluid being pumped as the collapsible reservoir is compressed.

50. The device of claim 49, wherein the actuator includes an anode and a cathode, a polymeric actuator material between the anode and cathode, an ionic separator membrane separating the anode or cathode from the polymer actuator material, an electrolyte and a flexible exterior housing, and/or wherein the actuator includes an activation mechanism selected from the group consisting of a spring, a piezo actuator, a nitenol or shape memory material, a hydraulic actuator, and an pneumatic actuator.

51. The device of claim 49, wherein the one or more sensors are selected from the group consisting of a motion sensor, a temperature sensor and a pressure sensor for sensing movement, temperature or pressure generated by the actuator, and optionally wherein one or more of the sensors provide external feedback from the patient to the controller.

52. The device of claim 49, further comprising a rigid plate located between the actuator and the fluid reservoir.

53. The device of claim 52, wherein the rigid plate has one or more hinges or flexure arms, and/or optionally further including a magnet located on the plate in close proximity to a magnorestrictive sensor to measure movement of the plate.

54. The device of claim 49, characterized by one or more of the following features:
(a) wherein the outlet valve is controlled to open and close from changes in pressure;
(b) wherein the outlet valve is electrically controlled via the controller;
(c) comprising one or more actuators and one or more reservoirs to hold and dispense one or more medications;
(d) wherein the inlet comprises a rigid inlet fluid channel with one or more bends sufficient to impede access by a needle, preferably totaling at least 90° in fluid connectivity between the inlet and the inlet valve;
(e) wherein the inlet valve is a one way anti siphon valve; and
(f) wherein the outlet comprises a rigid fluid channel with one or more bends of at least 90° in fluid connectivity between the reservoir and the outlet valve, and optionally, further comprising a second collapsible tube valve between the reservoir and the outlet valve.

55. A low profile medication delivery device comprising:
a chassis;
an actuator overlaying a collapsible reservoir disposed within the chassis, the collapsible reservoir having an inlet and outlet that exit the chassis;
wherein the actuator is configured to expand and compress the collapsible reservoir in response to electrical current being passed through the actuator;
a user interface for providing input to the electronics disposed within the chassis in electrical connection with the actuator, and one or more sensors electrically connected to the electronics;
a power supply in electrical connection with the electronics, and a controller to control the pump, sensors and electronics,
and a septum and valve in fluid connection with the reservoir inlet and one or more outlet valves in fluid connection with the reservoir outlet to control the fluid being pumped as the collapsible reservoir is compressed.

56. The device of claim 55, wherein the user interface is adapted to provide input information to the controller and visual information to the user, and/or wherein the controller is programmable and the program includes software hardrails that prevent the device pumping or dispensing a lethal dose of medication to the patient, whether the lethal dose is accumulated over time or all at once.

57. They device of claim 55, wherein the actuator includes an anode and a cathode, a polymeric actuator material between the anode and cathode, an ionic separator membrane separating the anode or cathode from the polymer actuator material, an electrolyte and a flexible exterior housing, and/or wherein the actuator includes an actuation mechanism selected from the group consisting of a spring, a piezo actuator, a nitenol or shape memory material, a hydraulic actuator, and a pneumatic actuator.

58. The device of claim 55, wherein the one or more sensors are selected from the group consisting of a motion sensor, a temperature sensor and a pressure sensor, and optionally wherein one or more of the sensors provide external feedback from the patient to the controller.

59. The device of claim 55, further including a rigid plate located between the actuator and the fluid reservoir.

60. The device of claim 59, wherein the rigid plate has one or more hinges or flexure arms, and/or optionally further including a magnet located on the plate in close proximity to a magnorestrictive sensor to measure movement of the plate.

61. The device of claim 55, characterized by one or more of the following features:
(a) wherein the outlet valve is controlled to open and close from changes in pressure;
(b) wherein the outlet valve is electrically controlled via the controller;
(c) comprising one or more actuators and one or more reservoirs to hold and dispense one or more medications;
(d) wherein the inlet comprises a rigid inlet fluid channel with one or more bends sufficient to impede access by a needle, preferably totaling at least 90° in fluid connectivity between the inlet and the inlet valve;
(e) wherein the inlet valve is a one way anti siphon valve;
(f) wherein the outlet comprises a rigid fluid channel with one or more bends sufficient to impede access by a needle, preferably totaling at least 90° in fluid connectivity between the reservoir and the outlet valve, and optionally further comprising a second collapsible tube valve between the reservoir and the outlet valve.
